(19) United States
(12) Patent Application Publication
(10) Pub. No.: US 2017/0005277 A1
(43) Pub. Date: Jan. 5, 2017

(54) COMPOUND, COMPOSITION AND ORGANIC LIGHT-EMITTING DEVICE

(71) Applicants: Cambridge Display Technology Limited, Godmanchester (GB); Sumitomo Chemical Company Limited, Tokyo (JP)

(72) Inventors: Matthew Stevenson, Godmanchester (GB); Simon King, Godmanchester (GB); Florence Bourcet, Godmanchester (GB)

(73) Assignees: Cambridge Display Technology Limited, Godmanchester (GB); Sumitomo Chemical Company Limited, Tokyo (JP)

(21) Appl. No.: 15/182,056

(22) Filed: Jun. 14, 2016

(30) Foreign Application Priority Data
Jun. 30, 2015 (GB) 1511462.2

(51) Int. Cl. H01L 51/00; C07F 7/08; C07D 409/14; C09K 11/06; C09K 11/02

(52) U.S. Cl. CPC H01L 51/0072; C09K 11/06; H01L 51/0085; C09K 11/025; C07D 409/14; H01L 51/0074; H01L 51/0067; C07F 7/0814; H01L 51/0094; H01L 51/5004

(57) ABSTRACT

A compound of formula (I)

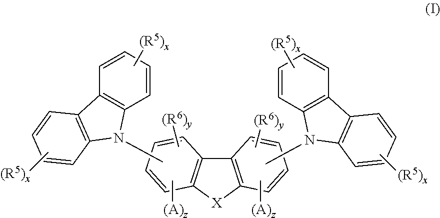

wherein X is O or S; each A is a LUMO-deepening substituent; R⁵ and R⁶ are independently in each occurrence a substituent; x independently in each occurrence is 0, 1, 2, 3 or 4; y independently in each occurrence is 0, 1 or 2, and each z is independently 0 or 1 with the proviso that at least one z is 1. The compound may be used as a host for a phosphorescent light-emitting material in an organic light-emitting device.

### RELATED APPLICATIONS

This application claims the benefits under 35 U.S.C. §119(a)-(d) or 35 U.S.C. §365(b) of British application number GB 1511462.2, filed Jun. 30, 2016, the entirety of which is incorporated herein.

### BACKGROUND OF THE INVENTION

Electronic devices containing active organic materials are attracting increasing attention for use in devices such as organic light emitting diodes (OLEDs), organic photoresponsive devices (in particular organic photovoltaic devices and organic photosensors), organic transistors and memory array devices. Devices containing active organic materials offer benefits such as low weight, low power consumption and flexibility. Moreover, use of soluble organic materials allows use of solution processing in device manufacture, for example inkjet printing or spin-coating.

An OLED may comprise a substrate carrying an anode, a cathode and one or more organic light-emitting layers between the anode and cathode.

Holes are injected into the device through the anode and electrons are injected through the cathode during operation of the device. Holes in the highest occupied molecular orbital (HOMO) and electrons in the lowest unoccupied molecular orbital (LUMO) of a light-emitting material combine to form an exciton that releases its energy as light.

Light-emitting materials include small molecule, polymeric and dendrimeric materials. Light-emitting polymers include poly(arylene vinylenes) such as poly(p-phenylene vinylenes) and polymers containing arylene repeat units, such as fluorene repeat units.

A light emitting layer may comprise a host material and a light-emitting dopant wherein energy is transferred from the host material to the light-emitting dopant. For example, J. Appl. Phys. 65, 3610, 1989 discloses a host material doped with a fluorescent light-emitting dopant (that is, a light-emitting material in which light is emitted via decay of a singlet exciton).

Phosphorescent dopants are also known (that is, a light-emitting dopant in which light is emitted via decay of a triplet exciton).

Sook et al, J. Mater. Chem., 2011, 21, 14604 discloses host materials DBT1, DBT2 and DBT3:

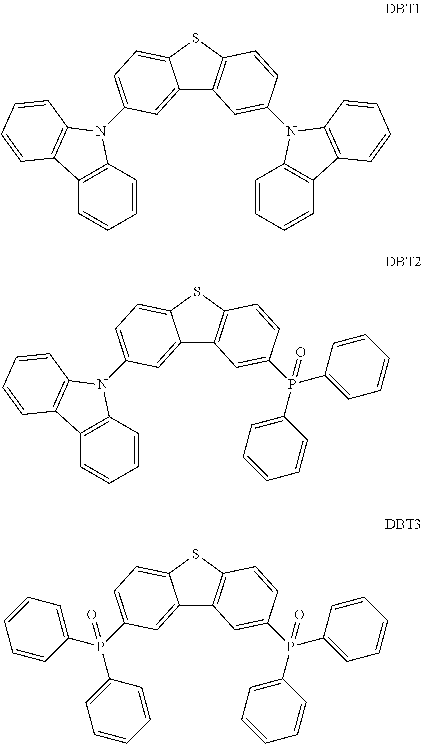 [SMILES: c1ccc2c(c1)c1ccccc1n2-c1ccc2sc3ccc(-n4c5ccccc5c5ccccc54)cc3c2c1 ; O=P(c1ccccc1)(c1ccccc1)c1ccc2sc3ccc(-n4c5ccccc5c5ccccc54)cc3c2c1 ; O=P(c1ccccc1)(c1ccccc1)c1ccc2sc3ccc(P(=O)(c4ccccc4)c4ccccc4)cc3c2c1]

U.S. Pat. No. 6,562,982 discloses hosts having the following formula wherein Ar is an aryl and R¹, R², R³ and R⁴ are independently a hydrocarbyl:

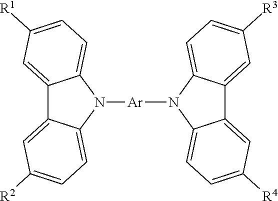

US 2013/0306940 discloses use of the compound DBT2Cz as a host for iridium complexes with certain carbene ligands:

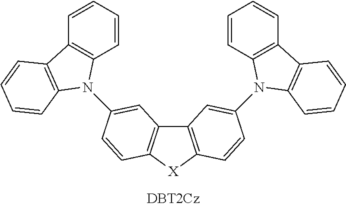

### SUMMARY OF THE INVENTION

In a first aspect the invention provides a compound of formula (I)

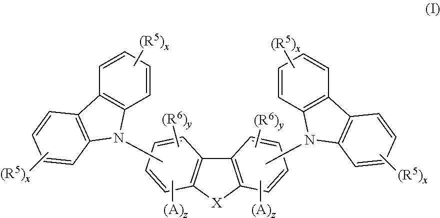

wherein:

X is O or S;

each A is a LUMO-deepening substituent;

R⁵ and R⁶ are independently in each occurrence a substituent;

x independently in each occurrence is 0, 1, 2, 3 or 4;

y independently in each occurrence is 0, 1 or 2, and

each z is independently 0 or 1 with the proviso that at least one z is 1.

In a second aspect the invention provides a composition comprising a compound according to the first aspect and at least one light-emitting material.

In a third aspect the invention provides a formulation comprising a compound according to the first aspect or a composition according to the second aspect and one or more solvents.

In a fourth aspect the invention provides an organic light-emitting device comprising an anode, a cathode and a light-emitting layer between the anode and the cathode wherein the light-emitting layer comprises a compound according to the first aspect.

In a fifth aspect the invention provides a method of forming an organic light-emitting device according to the fourth aspect, the method comprising the step of forming the light-emitting layer over one of the anode and the cathode and forming the other of the anode and the cathode over the light-emitting layer.

In a sixth aspect the invention provides a polymer having a polymer backbone and comprising a group of formula (II) in the polymer backbone, as a side-chain of the polymer backbone or and end group of the polymer backbone:

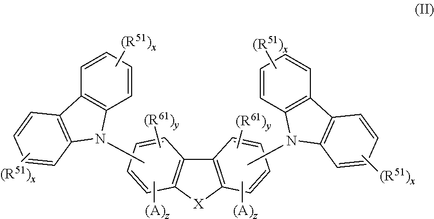

wherein X is O or S;

each A is independently a LUMO-deepening substituent;

R⁵¹ and R⁶¹ are independently in each occurrence a substituent;

x independently in each occurrence is 0, 1, 2, 3 or 4; and

y independently in each occurrence is 0, 1 or 2,

each z is independently 0 or 1 with the proviso that at least one z is 1, and

at least one of R⁵¹, R⁶¹ and A is bound to the polymer backbone.

### DESCRIPTION OF THE DRAWINGS

The invention will now be described in more detail with reference to the drawings in which:

FIG. 1 illustrates an OLED according to an embodiment of the invention.

### DETAILED DESCRIPTION OF THE INVENTION

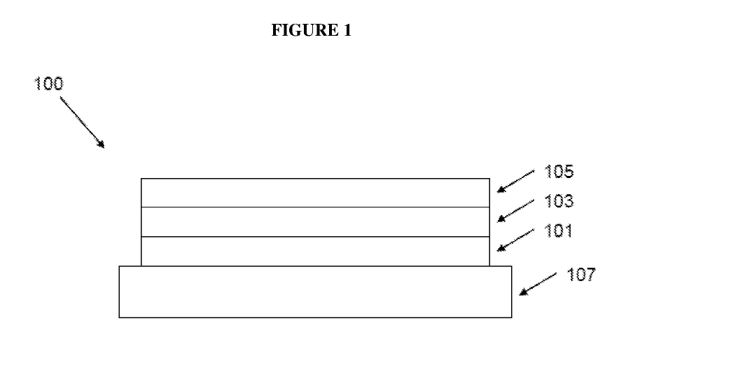

FIG. 1 illustrates an OLED 100 according to an embodiment of the invention comprising an anode 101, a cathode 105 and a light-emitting layer 103 between the anode and cathode. The device 100 is supported on a substrate 107, for example a glass or plastic substrate.

Light-emitting layer 103 may be unpatterned, or may be patterned to form discrete pixels. Each pixel may be further divided into subpixels. The light-emitting layer may contain a single light-emitting material, for example for a monochrome display or other monochrome device, or may contain materials emitting different colours, in particular red, green and blue light-emitting materials for a full-colour display.

One or more further layers may be provided between the anode 101 and cathode 105, for example hole-transporting layers, electron transporting layers, hole blocking layers and electron blocking layers. The device may contain more than one light-emitting layer.

Preferred device structures include:

Anode/Hole-injection layer/Light-emitting layer/Cathode

Anode/Hole transporting layer/Light-emitting layer/Cathode

Anode/Hole-injection layer/Hole-transporting layer/Light-emitting layer/Cathode

Anode/Hole-injection layer/Hole-transporting layer/Light-emitting layer/Electron-transporting layer/Cathode.

Preferably, at least one of a hole-transporting layer and hole injection layer is present. Preferably, both a hole injection layer and hole-transporting layer are present.

Light-emitting layer 103 may contain a composition of a compound of formula (I) doped with one or more luminescent dopants. The light-emitting layer 103 may consist essentially of these materials or may contain one or more further materials, for example one or more charge-transporting materials or one or more further light-emitting materials. Optionally, the light-emitting layer comprises a hole-transporting metal complex. The hole-transporting metal complex preferably has a HOMO level that is within 0.2 eV of the compound of formula (I). When used as a host material for one or more light-emitting dopants, the lowest excited stated singlet (S¹) in the case of a fluorescent dopant, or the lowest excited state triplet (T¹) energy level in the case of a phosphorescent dopant, of the compound of formula (I) is preferably no more than 0.1 eV below that of the light-emitting material, and is more preferably about the same as or higher than that of the light-emitting material in order to avoid quenching of luminescence from the light-emitting dopant. A charge transporting material present in the light-emitting layer prefer has a S¹ or T¹ higher than that of the light-emitting material.

Light-emitting materials include, without limitation, red, green and blue light-emitting materials.

Preferably, light-emitting layer 103 comprises a compound of formula (I) and a blue light-emitting dopant.

In the case where the luminescent dopant is a phosphorescent dopant, the compound of formula (I) preferably has a T¹ of greater than 2.8 eV, preferably greater than 3.0 eV.

The triplet energy level of a material may be measured from the energy onset of its phosphorescence spectrum measured by low temperature phosphorescence spectroscopy (Y. V. Romaovskii et al, Physical Review Letters, 2000, 85 (5), p1027, A. van Dijken et al, Journal of the American Chemical Society, 2004, 126, p 7718).

Preferably, light-emitting layer comprises a compound of formula (I) and a light-emitting material, preferably a blue light-emitting material, more preferably a blue phosphorescent material wherein the LUMO of the compound of formula (I) is shallower (closer to vacuum) than that of the light-emitting dopant. Preferably, the LUMO of the compound of formula (I) is less than 0.5 eV shallower than that of the light-emitting material, optionally no more than 0.4 or 0.3 eV shallower.

OLED 100 may contain more than one light-emitting material, preferably a plurality of light-emitting materials that together provide white light emission.

A white-emitting OLED may contain a single, white-emitting layer containing a light-emitting composition comprising a compound of formula (I) and light-emitting materials that produce which light, or may contain two or more layers that emit different colours which, in combination, produce white light and wherein at least one of the light emitting layers contains a composition comprising a compound of formula (I) and at least one light-emitting dopant. Optionally, the device comprises a red light-emitting layer and a green and blue light-emitting layer.

A blue emitting material may have a photoluminescent spectrum with a peak in the range of 400-490 nm, optionally 420-490 nm.

A green emitting material may have a photoluminescent spectrum with a peak in the range of more than 490nm up to 580 nm, optionally more than 490 nm up to 540 nm.

A red emitting material may optionally have a peak in its photoluminescent spectrum of more than 580 nm up to 630 nm, optionally 585-625 nm.

The photoluminescence spectrum of a light-emitting material as described herein may be measured by casting 5 wt % of the material in a PMMA film onto a quartz substrate to achieve transmittance values of 0.3-0.4 and measuring in a nitrogen environment using apparatus C9920-02 supplied by Hamamatsu.

The light emitted from a white-emitting OLED may have CIE x coordinate equivalent to that emitted by a black body at a temperature in the range of 2500-9000K and a CIE y coordinate within 0.05 or 0.025 of the CIE y co-ordinate of said light emitted by a black body, optionally a CIE x coordinate equivalent to that emitted by a black body at a temperature in the range of 2700-6000K.

The substituent A of formula (I) is a LUMO-deepening substituent. By “LUMO deepening substituent” is meant a substituent that results in a compound of formula (I) having a LUMO level that is deeper (further from vacuum) than a corresponding compound in which the substituent A is replaced with H.

The compound of formula (I) preferably has a LUMO level that is more than 2.00 eV from vacuum level, optionally more than 2.10 eV or more than 2.20 eV from vacuum level, wherein the LUMO level is measured by square wave voltammetry as described herein.

Optionally, each A is independently selected from the group consisting of:

(Ar¹)ₚ wherein Ar¹ independently in each occurrence is a C₆₋₂₀ aryl or 5-20 membered heteroaryl group that may be unsubstituted or substituted with one or more substituents, and p is 1 or more, optionally 1, 2 or 3; and

SiR¹₃ wherein each R¹ independently is selected from the group consisting of: alkyl, optionally C₁₋₂₀ alkyl, wherein one or more non-adjacent C atoms may be replaced with optionally substituted aryl or heteroaryl, O, S, substituted N, C═O or —COO—; and (Ar²)_{q} wherein Ar² independently in each occurrence is a C₆₋₂₀ aryl or heteroaryl group that may be unsubstituted or substituted with one or more substituents, and q is at least 1, optionally 1, 2 or 3.

Preferably, Ar¹ is phenyl or a 5-20 membered heteroaryl

Optionally, the heteroatoms of a heteroaromatic group Ar¹ are selected from N, O, S and combinations thereof. Preferably, heteroaryl groups Ar¹ are selected from 6-membered heteroaryl groups of C and N atoms that may be unfused or may be fused to one or more further C₆₋₂₀ aromatic or 5-20 membered heteroaromatic groups. Preferably, heteroaryl groups Ar¹ are selected from pyridine, diazines, optionally pyrimidine or pyrazine and triazines, preferably pyrimidine.

Each group Ar¹ may be unsubstituted or substituted with one or more substituents. Optionally, substituents are selected from R² wherein R² in each occurrence is independently C₁₋₂₀ alkyl wherein one or more non-adjacent C atoms may be replaced O, S, C═O or —COO—.

Preferably, the, or at least one, Ar¹ group of (Ar¹)ₚ is substituted with at least one substituent, preferably a C₁₋₂₀ alkyl group.

Preferably, R¹ is (Ar²)_{q}.

Preferably Ar² is phenyl.

Preferably, q is 1.

Each Ar² group may be unsubstituted or substituted with one or more substituents. Optionally, substituents for Ar² are R² wherein R² in each occurrence is independently selected from C₁₋₂₀ alkyl wherein one or more non-adjacent C atoms may be replaced O, S, C═O or —COO—, and one or more H atoms may be replaced with F.

Exemplary groups A are illustrated below:

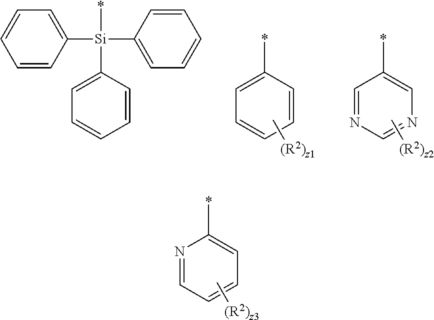

Wherein R² is as described above; z1 is 0, 1, 2, 3, 4 or 5, preferably at least 1; z2 is 0, 1, 2 or 3, preferably at least 1; and z3 is 0, 1, 2, 3 or 4, preferably at least 1.

The compound of formula (I) may have formula (Ia):

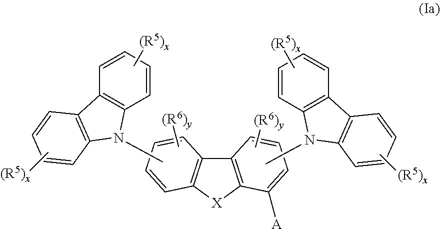

Each R⁵ and R⁶ of formula (I), where present, may independently in each occurrence be selected from the group consisting of alkyl, optionally C₁₋₂₀ alkyl, wherein one or more non-adjacent C atoms may be replaced with optionally substituted aryl or heteroaryl, O, S, substituted N, C═O or —COO—; aryl and heteroaryl groups that may be unsubstituted or substituted with one or more substituents, preferably phenyl substituted with one or more C₁₋₂₀ alkyl groups; F; CN and NO₂.

Preferably, each x is 0.

Preferably, each y is 0.

Exemplary compounds of formula (I) include the following:

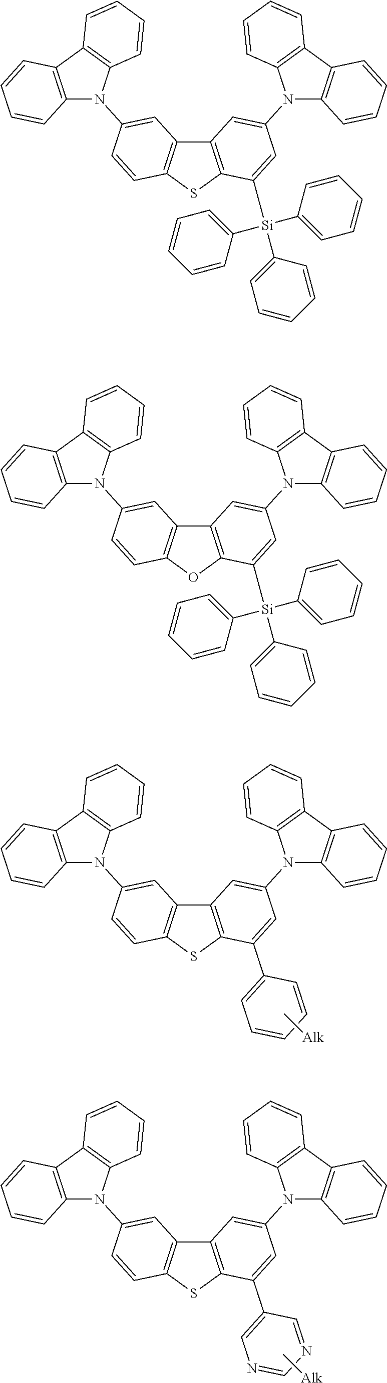

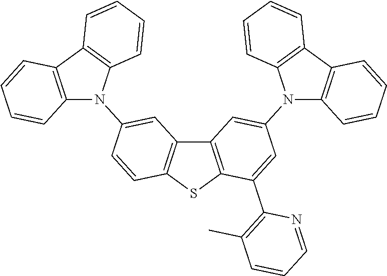 [SMILES: Cc1cccnc1-c1cc(-n2c3ccccc3c3ccccc32)cc2c1sc1ccc(-n3c4ccccc4c4ccccc43)cc12]

wherein Alk represents one or more C₁₋₂₀ alkyl groups.

The compound of formula (I) may be provided in a polymer as a repeating unit in a backbone of the polymer, as a side-group pendant from the polymer backbone or and an end-group of the polymer backbone.

The polymer may comprise a group of formula (II):

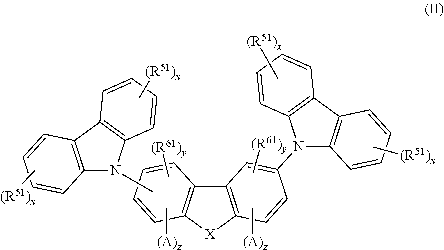

wherein X, A, x, y and z are as described herein, and R⁵¹ and R⁶¹ are as described with reference to R⁵ and R⁶ respectively.

Light-Emitting Compounds

A preferred use of compounds of formula (I) is as the host material for a light-emitting material in a light-emitting layer of an OLED.

Suitable light-emitting materials for a light-emitting layer include polymeric, small molecule and dendritic light-emitting materials, each of which may be fluorescent or phosphorescent.

Preferred phosphorescent compounds are second or third row transition metal complexes, preferably complexes of ruthenium, rhodium, palladium, rhenium, osmium, iridium, platinum or gold. Iridium is particularly preferred.

The compound of formula (I) may be provided in a composition comprising one, two or more light-emitting compounds. Preferably, the composition comprises at least one phosphorescent compound having a LUMO at least 2.2 or 2.3 eV from vacuum level. Optionally, the transition metal complex comprises at least one ligand, preferably a bidentate ligand, comprising a carbene group coordinated to the metal. Preferably, transition metal complexes comprising a carbene group are blue light-emitting materials.

Optionally, the phosphorescent compound has formula (IX):

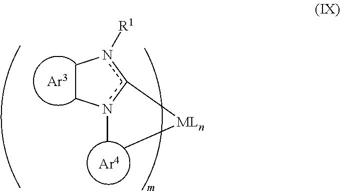

wherein M is a second or third row transition metal;

m is at least 1, optionally 1, 2 or 3;

n is 0 or a positive integer;

R¹ is H or a substituent;

Ar³ and Ar⁴ are each independently a monocyclic or fused aryl or heteroaryl group; and

L is a ligand that does not comprise a carbene group.

Optionally, R¹ is selected from the group consisting of C₁₋₂₀ alkyl and a group of formula —(Ar⁵)ₜ wherein Ar⁵ is a C₆₋₂₀ aryl group or 5-20 membered heteroaryl group and t is at least 1, optionally 1, 2 or 3.

Ar⁵ is preferably phenyl.

Each Ar⁵ may independently be unsubstituted or substituted with one or more substituents, optionally one or more C₁₋₂₀ alkyl groups.

Ar³ is preferably a monocyclic heteroaromatic group of C and N atoms, optionally pyridine or pyrazine.

Ar⁴ is preferably a C₆₋₂₀ aryl group, more preferably phenyl.

Ar³ and Ar⁴ may each independently be unsubstituted or substituted with one or more substituents R³ wherein each R³ is independently selected from the group consisting of:

D;

F;

CN;

C₁₋₂₀ alkyl, wherein one or more non-adjacent C atoms may be replaced with optionally substituted aryl or heteroaryl (preferably phenyl), O, S, C═O or —COO—, and one or more H atoms may be replaced with F; and

—(Ar⁵)ₜ as described above.

Exemplary ligands L are N,N-bidentate ligands, optionally bipyridyl; N,O-bidentate ligands, optionally picolinate; and O,O-bidentate ligands, optionally acac.

Optionally, the group of formula (IX) has formula (IXa):

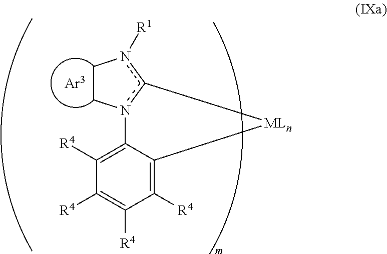

wherein each R⁴ is independently H or a substituent R³ as described above.

Optionally, the group of formula (IX) has formula (IXb):

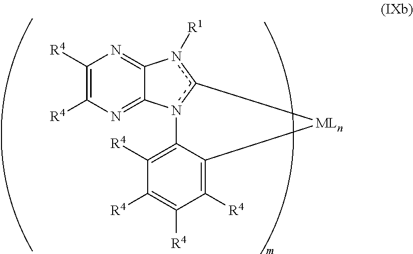

Optionally, the phosphorescent compound has formula (III):

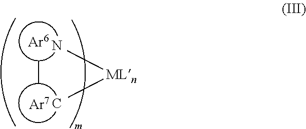

wherein M, m and n are as described with reference to Formula (IX); L′ is a ligand other than a C,N-cyclometallating ligand; Ar⁷ is a 5-20 membered aryl or heteroaryl group that may be unsubstituted or substituted with one or more substituents; and Ar⁶ is a 5-20 membered heteroaryl group that may be unsubstituted or substituted with one or more substituents. Optionally, compounds of formula (III) are green or red phosphorescent materials.

Preferably, Ar⁶ is a 5-20 membered heteroaryl group of C and N atoms, optionally pyridine or quinoline.

Preferably, Ar⁷ is a 5-20 membered aryl group, optionally phenyl.

Each of Ar⁶ and Ar⁷ is independently unsubstituted or substituted with one or more substituents. Substituents may be selected from: F; —(Ar¹)_{q} wherein Ar¹ independently in each occurrence is a C₆₋₂₀ aryl or 5-20 membered heteroaryl group that may be unsubstituted or substituted with one or more substituents and q is 1 or more, optionally 1, 2 or 3; and C₁₋₂₀ alkyl wherein one or more non-adjacent C atoms may be replaced O, S, C═O or —COO and one or more H atoms may be replaced with F.

Each Ar¹ is independently unsubstituted or substituted with one or more substituents, preferably one or more C₁₋₂₀ alkyl groups.

The compound of formula (III) may be a light-emitting dendrimer comprising one or more dendrons bound to Ar⁶ and/or Ar⁷, wherein each dendron comprises a branching point and two or more dendritic branches. Preferably, the dendron is at least partially conjugated, and at least one of the branching points and dendritic branches comprises an aryl or heteroaryl group, for example a phenyl group or 6-membered heteroaromatic of C and N atoms, optionally triazine. In one arrangement, the branching point group and the branching groups are all phenyl, and each phenyl may independently be substituted with one or more substituents, for example alkyl or alkoxy.

A dendron may have optionally substituted formula (XII)

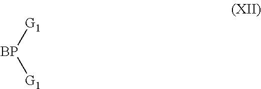

wherein BP represents a branching point for attachment to a core and G₁ represents first generation branching groups.

The dendron may be a first, second, third or higher generation dendron. G₁ may be substituted with two or more second generation branching groups G₂, and so on, as in optionally substituted formula (XIIa):

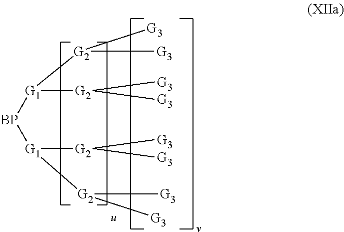

wherein u is 0 or 1; v is 0 if u is 0 or may be 0 or 1 if u is 1; BP represents a branching point for attachment to a core and G₁, G₂ and G₃ represent first, second and third generation dendron branching groups. In one preferred embodiment, each of BP and G₁, G₂ . . . Gₙ is phenyl, and each phenyl BP, G₁, G₂ . . . Gₙ₋₁ is a 3,5-linked phenyl.

Preferred dendrons are substituted or unsubstituted dendrons of formulae (XIIb) and (XIIc):

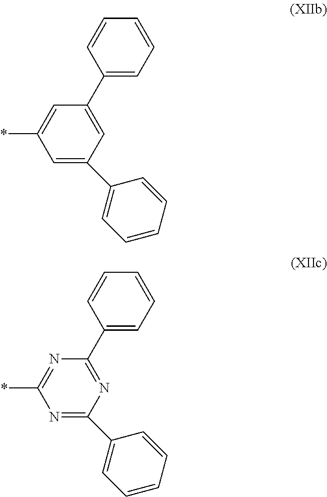

wherein * represents an attachment point of the dendron to Ar⁶ or Ar⁷.

BP and/or any group G may be substituted with one or more substituents, for example one or more C₁₋₂₀ alkyl or alkoxy groups.

Light-emitting material(s) in a composition comprising the compound of formula (I) and one or more light-emitting materials may make up about 0.05 wt % up to about 50 wt %, optionally about 1-40 wt % of the composition.

Charge Transporting and Charge Blocking Layers

A device containing a light-emitting layer containing a compound of formula (I) may have charge-transporting and/or charge blocking layers.

A hole transporting layer may be provided between the anode and the light-emitting layer or layers of an OLED. An electron transporting layer may be provided between the cathode and the light-emitting layer or layers.

An electron blocking layer may be provided between the anode and the light-emitting layer(s) and a hole blocking layer may be provided between the cathode and the light-emitting layer(s). Charge-transporting and charge-blocking layers may be used in combination. Depending on the HOMO and LUMO levels of the material or materials in a layer, a single layer may both transport one of holes and electrons and block the other of holes and electrons.

If present, a hole transporting layer located between the anode and the light-emitting layer(s) preferably has a material having a HOMO level of less than or equal to 5.5 eV, more preferably around 4.8-5.5 eV or 4.9-5.3 eV as measured by cyclic voltammetry.

The HOMO level of the material in the hole transport layer may be selected so as to be within 0.2 eV, optionally within 0.1 eV of the light-emitting material of the light-emitting layer.

A hole-transporting layer may contain polymeric or non-polymeric charge-transporting materials. Exemplary hole-transporting materials contain arylamine groups.

A hole transporting layer may contain a homopolymer or copolymer comprising a repeat unit of formula (VII):

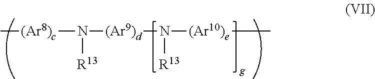

wherein Ar⁸, Ar⁹ and Ar¹⁰ in each occurrence are independently selected from substituted or unsubstituted aryl or heteroaryl, g is 0, 1 or 2, preferably 0 or 1, R¹³ independently in each occurrence is H or a substituent, preferably a substituent, and c, d and e are each independently 1, 2 or 3.

R¹³, which may be the same or different in each occurrence when g is 1 or 2, is preferably selected from the group consisting of alkyl, for example C₁₋₂₀ alkyl, Ar¹¹, a branched or linear chain of Ar¹¹ groups, or a crosslinkable unit that is bound directly to the N atom of formula (I) or spaced apart therefrom by a spacer group, wherein Ar¹¹ in each occurrence is independently optionally substituted aryl or heteroaryl. Exemplary spacer groups are C₁₋₂₀ alkyl, phenyl and phenyl-C₁₋₂₀ alkyl.

Any two aromatic or heteroaromatic groups selected from Ar⁸, Ar⁹, and, if present, Ar¹⁰ and Ar¹¹ directly bound to the same N atom may be linked by a direct bond or a divalent linking atom or group to another of Ar⁸, Ar⁹, Ar¹⁰ and Ar¹¹. Preferred divalent linking atoms and groups include O, S; substituted N; and substituted C.

Ar⁸ and Ar¹⁰ are preferably C₆₋₂₀ aryl, more preferably phenyl, that may be unsubstituted or substituted with one or more substituents.

In the case where g=0, Ar⁹ is preferably C₆₋₂₀ aryl, more preferably phenyl, that may be unsubstituted or substituted with one or more substituents.

In the case where g=1, Ar⁹ is preferably C₆₋₂₀ aryl, more preferably phenyl or a polycyclic aromatic group, for example naphthalene, perylene, anthracene or fluorene, that may be unsubstituted or substituted with one or more substituents.

R¹³ is preferably Ar¹¹ or a branched or linear chain of Ar¹¹ groups. Ar¹¹ in each occurrence is preferably phenyl that may be unsubstituted or substituted with one or more substituents.

Exemplary groups R¹³ include the following, each of which may be unsubstituted or substituted with one or more substituents, and wherein * represents a point of attachment to N:

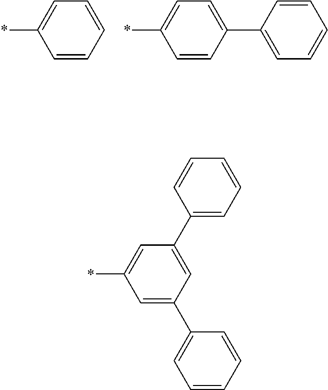

c, d and e are preferably each 1.

Ar⁸, Ar⁹, and, if present, Ar¹⁰ and Ar¹¹ are each independently unsubstituted or substituted with one or more, optionally 1, 2, 3 or 4, substituents. Exemplary substituents may be selected from:
substituted or unsubstituted alkyl, optionally C₁₋₂₀ alkyl, wherein one or more non-adjacent C atoms may be replaced with optionally substituted aryl or heteroaryl (preferably phenyl), O, S, C═O or —COO— and one or more H atoms may be replaced with F; and
a crosslinkable group attached directly to or forming part of Ar⁸, Ar⁹, Ar¹⁰ or Ar¹¹ or spaced apart therefrom by a spacer group, for example a group comprising a double bond such and a vinyl or acrylate group, or a benzocyclobutane group.

Preferred substituents of Ar⁸, Ar⁹, and, if present, Ar¹⁰ and Ar¹¹ are C₁₋₄₀ hydrocarbyl, preferably C₁₋₂₀ alkyl or a hydrocarbyl crosslinking group.

Preferred repeat units of formula (VII) include units of formulae 1-3:

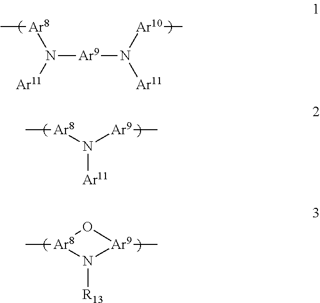

Preferably, Ar⁸, Ar¹⁰ and Ar¹¹ of repeat units of formula 1 are phenyl and Ar⁹ is phenyl or a polycyclic aromatic group.

Preferably, Ar⁸, Ar⁹ and Ar¹¹ of repeat units of formula 2 are phenyl.

Preferably, Ar⁸ and Ar⁹ of repeat units of formula 3 are phenyl and Ar¹¹ is phenyl or a branched or linear chain of phenyl groups.

A polymer comprising repeat units of formula (VII) may be a homopolymer or a copolymer containing repeat units of formula (VII) and one or more co-repeat units.

In the case of a copolymer, repeat units of formula (VII) may be provided in a molar amount in the range of about 1-99 mol %, optionally about 1-50 mol %.

Exemplary co-repeat units include arylene repeat units that may be unsubstituted or substituted with one or more substituents, for example one or more C₁₋₄₀ hydrocarbyl groups.

Exemplary arylene repeat units include without limitation, fluorene, phenylene, naphthalene, anthracene, indenofluorene, phenanthrene and dihydrophenanthrene repeat units, each of which may be unsubstituted or substituted with one or more substituents.

Substituents of arylene repeat units, if present, may be selected from C₁₋₄₀ hydrocarbyl, preferably C₁₋₂₀ alkyl; phenyl which may be unsubstituted or substituted with one or more C₁₋₁₀ alkyl groups; and crosslinkable hydrocarbyl groups, for example C₁₋₄₀ hydrocarbyl groups comprising benzocyclobutene or vinylene groups.

Phenylene repeat units may be 1,4-linked phenylene repeat units that may be unsubstituted or substituted with 1, 2, 3 or 4 substituents. Fluorene repeat units may be 2,7-linked fluorene repeat units.

Fluorene repeat units preferably have two substituents in the 9-position thereof. Aromatic carbon atoms of fluorene repeat units may each independently be unsubstituted or substituted with a substituent.

If present, an electron transporting layer located between the light-emitting layers and cathode preferably has a LUMO level of around 1.8-2.7 eV as measured by cyclic voltammetry. An electron-transporting layer may have a thickness in the range of about 5-50 nm.

A charge-transporting layer or charge-blocking layer may be crosslinked, particularly if a layer overlying that charge-transporting or charge-blocking layer is deposited from a solution. The crosslinkable group used for this crosslinking may be a crosslinkable group comprising a reactive double bond such and a vinyl or acrylate group, or a benzocyclobutane group. The crosslinkable group may be provided as a substituent of, or may be mixed with, a charge-transporting or charge-blocking material used to form the charge-transporting or charge-blocking layer.

A charge-transporting layer adjacent to a light-emitting layer containing a phosphorescent light-emitting material preferably contains a charge-transporting material having a lowest triplet excited state (T₁) excited state that is no more than 0.1 eV lower than, preferably the same as or higher than, the T₁ excited state energy level of the phosphorescent light-emitting material(s) in order to avoid quenching of triplet excitons.

A charge-transporting layer as described herein may be non-emissive, or may contain a light-emitting material such that the layer is a charge transporting light-emitting layer. If the charge-transporting layer is a polymer then a light-emitting dopant may be provided as a side-group of the polymer, a repeat unit in a backbone of the polymer, or an end group of the polymer. Optionally, a hole-transporting polymer as described herein comprises a phosphorescent polymer in a side-group of the polymer, in a repeat unit in a backbone of the polymer, or as an end group of the polymer.

The polystyrene-equivalent number-average molecular weight (Mn) measured by gel permeation chromatography of the polymers described herein may be in the range of about 1×10³ to 1×10⁸, and preferably 1×10⁴ to 5×10⁶. The polystyrene-equivalent weight-average molecular weight (Mw) of the polymers described herein may be 1×10³ to 1×10⁸, and preferably 1×10⁴ to 1×10⁷.

Polymers as described herein are suitably amorphous.

Hole Injection Layers

A conductive hole injection layer, which may be formed from a conductive organic or inorganic material, may be provided between the anode 101 and the light-emitting layer 103 of an OLED as illustrated in FIG. 1 to assist hole injection from the anode into the layer or layers of semiconducting polymer. Examples of doped organic hole injection materials include optionally substituted, doped poly(ethylene dioxythiophene) (PEDOT), in particular PEDOT doped with a charge-balancing polyacid such as polystyrene sulfonate (PSS) as disclosed in EP 0901176 and EP 0947123, polyacrylic acid or a fluorinated sulfonic acid, for example Nafion®; polyaniline as disclosed in U.S. Pat. No. 5,723,873 and U.S. Pat. No. 5,798,170; and optionally substituted polythiophene or poly(thienothiophene). Examples of conductive inorganic materials include transition metal oxides such as VOx, MoOx and RuOx as disclosed in Journal of Physics D: Applied Physics (1996), 29(11), 2750-2753.

Cathode

The cathode 105 is selected from materials that have a workfunction allowing injection of electrons into the light-emitting layer of the OLED. Other factors influence the selection of the cathode such as the possibility of adverse interactions between the cathode and the light-emitting material. The cathode may consist of a single material such as a layer of aluminium. Alternatively, it may comprise a plurality of conductive materials such as metals, for example a bilayer of a low workfunction material and a high workfunction material such as calcium and aluminium, for examples disclosed in WO 98/10621. The cathode may comprise elemental barium, for example as disclosed in WO 98/57381, Appl. Phys. Lett. 2002, 81(4), 634 and WO 02/84759. The cathode may comprise a thin (e.g. 1-5 nm) layer of metal compound, in particular an oxide or fluoride of an alkali or alkali earth metal, between the organic layers of the device and one or more conductive cathode layers to assist electron injection, for example lithium fluoride as disclosed in WO 00/48258; barium fluoride as disclosed in Appl. Phys. Lett. 2001, 79(5), 2001; and barium oxide. In order to provide efficient injection of electrons into the device, the cathode preferably has a work function of less than 3.5 eV, more preferably less than 3.2 eV, most preferably less than 3 eV. Work functions of metals can be found in, for example, Michaelson, J. Appl. Phys. 48(11), 4729, 1977.

The cathode may be opaque or transparent. Transparent cathodes are particularly advantageous for active matrix devices because emission through a transparent anode in such devices is at least partially blocked by drive circuitry located underneath the emissive pixels. A transparent cathode comprises a layer of an electron injecting material that is sufficiently thin to be transparent. Typically, the lateral conductivity of this layer will be low as a result of its thinness. In this case, the layer of electron injecting material is used in combination with a thicker layer of transparent conducting material such as indium tin oxide.

It will be appreciated that a transparent cathode device need not have a transparent anode (unless, of course, a fully transparent device is desired), and so the transparent anode used for bottom-emitting devices may be replaced or supplemented with a layer of reflective material such as a layer of aluminum. Examples of transparent cathode devices are disclosed in, for example, GB 2348316.

Encapsulation

Organic optoelectronic devices tend to be sensitive to moisture and oxygen. Accordingly, the substrate preferably has good barrier properties for prevention of ingress of moisture and oxygen into the device. The substrate is commonly glass, however alternative substrates may be used, in particular where flexibility of the device is desirable. For example, the substrate may comprise one or more plastic layers, for example a substrate of alternating plastic and dielectric barrier layers or a laminate of thin glass and plastic.

The device may be encapsulated with an encapsulant (not shown) to prevent ingress of moisture and oxygen. Suitable encapsulants include a sheet of glass, films having suitable barrier properties such as silicon dioxide, silicon monoxide, silicon nitride or alternating stacks of polymer and dielectric or an airtight container. In the case of a transparent cathode device, a transparent encapsulating layer such as silicon monoxide or silicon dioxide may be deposited to micron levels of thickness, although in one preferred embodiment the thickness of such a layer is in the range of 20-300 nm. A getter material for absorption of any atmospheric moisture and/or oxygen that may permeate through the substrate or encapsulant may be disposed between the substrate and the encapsulant.

Formulation Processing

A formulation suitable for forming a light-emitting layer may be formed from a compound of formula (I), any further components of the layer such as light-emitting materials, and one or more suitable solvents.

The formulation may be a solution of the compound of formula (I) and any other components in the one or more solvents, or may be a dispersion in the one or more solvents in which one or more components are not dissolved. Preferably, the formulation is a solution.

Solvents suitable for dissolving compounds of formula (I) are solvents comprising alkyl substituents for example benzenes substituted with one or more C₁₋₁₀ alkyl or C₁₋₁₀ alkoxy groups, for example toluene, xylenes and methylanisoles.

Particularly preferred solution deposition techniques including printing and coating techniques such spin-coating, inkjet printing and slot-die coating.

Spin-coating is particularly suitable for devices wherein patterning of the light-emitting layer is unnecessary—for example for lighting applications or simple monochrome segmented displays.

Inkjet printing is particularly suitable for high information content displays, in particular full colour displays. A device may be inkjet printed by providing a patterned layer over the first electrode and defining wells for printing of one colour (in the case of a monochrome device) or multiple colours (in the case of a multicolour, in particular full colour device). The patterned layer is typically a layer of photoresist that is patterned to define wells as described in, for example, EP 0880303.

As an alternative to wells, the ink may be printed into channels defined within a patterned layer. In particular, the photoresist may be patterned to form channels which, unlike wells, extend over a plurality of pixels and which may be closed or open at the channel ends.

Other solution deposition techniques include dip-coating, roll printing and screen printing.

### EXAMPLES

### Compound Example 1

Compound Example 1 was prepared according to the following reaction scheme:

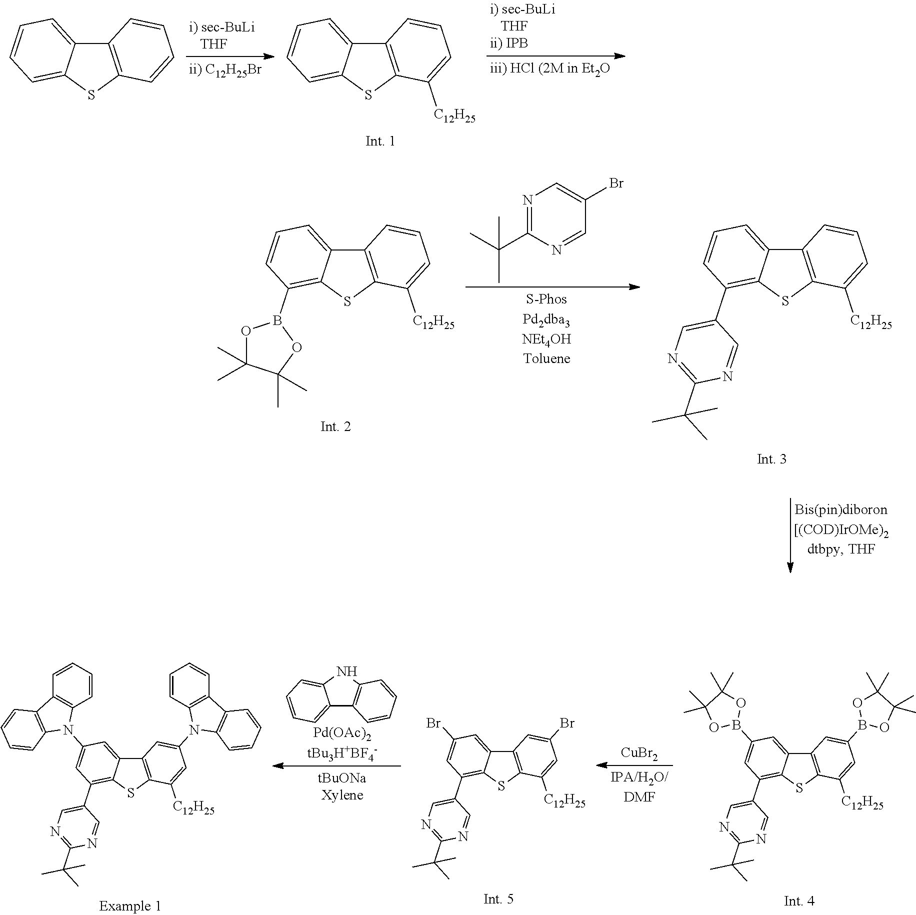

### 4-dodecyldibenzothiophene (Int. 1)

To a solution of dibenzothiophene (130.0 g, 705.5 mmol) in 1.3 L of anhydrous THF at −74° C. was added sec-BuLi (1.4M in cyclohexane, 650 ml, 917.2 mmol) drop wise such as internal temperature <−70° C. and the mixture was stirred for 2 h at −74° C. 1-Bromododecane (256 ml, 1058.9 mmol) in solution in 200 ml of anhydrous THF was added drop wise at −74° C. such as internal temperature <−70° C., and reaction mixture was allowed to warm up to room temperature overnight.

Reaction mixture was quenched with 500 ml of HCl (2M aqueous) added drop wise at 0° C. THF was removed under reduced pressure. Biphasic residue was extracted 3× with hexane; combined organic layers were washed 3× with water, dried over MgSO₄ and concentrated to dryness. Residue was purified by column chromatography in 3 batches. Fractions containing product were combined and concentrated to dryness under reduced pressure to give a white solid. It was dried in vacuum oven at 40° C. for 18 h to yield the product (Int. 1) as a white waxy solid (230 g, yield=92%).

### 2-(6-dodecyldibenzothiophen-4-yl)-4,4,5,5-tetramethyl-1,3,2-dioxaborolane (Int. 2)

To a slurry of Int. 1 (149.3 g, 423.5 mmol) in 2.25 L of anhydrous THF at −74° C. was added sec-BuLi (1.4M in cyclohexane, 456 ml, 638.2 mmol) drop wise such as internal temperature <−70° C. Reaction mixture was allowed to warm up to −40° C. and stirred for 2 h at −40° C. to give a dark brown solution. Mixture was cooled to −74° C. and isopropoxyboronic acid pinacol ester (139 ml, 680.7 mmol) was added drop wise at −74° C. such as internal temperature <−70° C. Reaction mixture was allowed to warm up to room temperature overnight. Mixture was cooled to −30° C. and HCl (2M in diethyl ether, 298 ml, 595.6 ml) was added to it. Mixture was allowed to warm up to room temperature and the solvents were removed under reduced pressure.

The residue was stirred with hexane and filtered. Filtrate was concentrated and filtered through a silica plug, eluted with hexane followed by a mixture of hexane and ethyl acetate (80:20). Fractions containing product were evaporated, and stirred in methanol at 0° C. The solid was filtered and washed several time with methanol. It was dried in vacuum oven at 40° C. for 72 h to yield the product (Int. 2) as a white powder (155.6 g, yield=77%).

### 2-(tert-butyl)-5-(6-dodecyldibenzothiophen-4-yl)pyrimidine (Int. 3)

Nitrogen was bubbled for 1.5 h into a mixture of Int. 2 (60.0 g, 125.4 mmol) and 5-bromo-2-(tert-butyl)pyrimidine (28.3 g, 131.7 mmol) in 960 ml of toluene. Meanwhile nitrogen was bubbled in a solution of tetrabutyl ammonium hydroxide (20% w/v in water, 370 ml, 501.5 mmol) for 45 min. Tris(dibenzylidene acetone) dipalladium (0.574 g, 0.65 mmol), followed by 2-dicyclohexylphosphino-2′,6′-dimethoxybiphenyl (0.564 g, 1.25 mmol) were added to the toluene solution. Mixture was heated up to 105° C. and the base was added drop wise into it. Reaction was stirred overnight at 105° C. and cooled down to room temperature. Phases were separated and aqueous layer was extracted 1× with toluene. Combined organic layers were washed 3× with water dried over magnesium sulphate and concentrated to dryness under reduced pressure. Residue was dissolved in a mixture of hexane and dichloromethane (50:50) and filtered through a silica/florisil plug (sinter funnel packed with a layer of florisil on top of a layer of silica), eluted with in a mixture of hexane and dichloromethane (50:50). The filtrate was concentrated under reduced pressure and dried in vacuum oven at 50° C. to yield the product (Int. 3) as a colorless oil (60 g, yield=98%).

### 2-(tert-butyl)-5-(6-dodecyl-2,8-bis(4,4,5,5-tetramethyl-1,3,2-dioxaborolan-2-yl)dibenzothiophen-4-yl)pyrimidine (Int. 4)

Nitrogen was bubbled for 1 h into a mixture of Int. 3 (57.5 g, 118.5 mmol) and bis(pinacolato)diboron (90.3 g, 355.6 mmol) in 580 ml of THF. 4,4′-di-tert-butyldipyridine (0.698 g, 2.37 mmol) followed by (1,5-cyclooctadiene) (methoxy)iridium (I) dimer (.786 g, 1.18 mmol) was added into the THF solution. Reaction was refluxed at 85° C. overnight. Mixture was allowed to cool down to room temperature and carefully poured into a beaker containing 2 L of methanol. Mixture was concentrate to a volume of 1 L, resulting slurry was stirred for 1 h and filtered.

The solid was recrystallized 4× from hexane and 1× from a mixture of toluene and acetonitrile. It was dried in vacuum oven at 50° C. to yield the product (Int. 4) as a white powder (56.2 g, yield=64%). HPLC indicated a purity of 98.7%.

### 2-(tert-butyl)-5-(2,8-dibromo-6-dodecyldibenzo[b,d]thiophen-4-yl)pyrimidine (Int. 5)

To a solution of Int. 4 (30.0 g, 40.6 mmol) in a mixture of 565 ml of propan-2-ol, 565 1 of N,N-dimethylformamide and 677 ml of water at 110° C. was added copper (II) bromide (108.8 g, 487.4 mmol) in 4 portions over 4 h. Mixture was stirred overnight at 110° C. Extra copper (II) bromide (100.0 g, 447.7 mmol) was added in 4 portions over 7 h and mixture was stirred overnight at 110° C. It was allowed to cool down to room temperature and filtered. Solid was extracted 5× with 200 ml of ethyl acetate. Combined organics were washed 3× with water, dried over magnesium sulphate and concentrated under reduced pressure. Residue was purified by column chromatography on silica eluting with a mixture of hexane and ethyl acetate (90:10). Fractions containing product were combined and concentrated under reduced pressure. Residue was stirred with methanol and solvent was decanted. The resulting oil was dried in vacuum oven at 50° C. to yield the product (Int. 5) as a colorless oil (16.7 g, yield=64%). HPLC indicated a purity of 99.3%.

### 9,9′-(4-(2-(tert-butyl)pyrimidin-5-yl)-6-dodecyldibenzo[b,d]thiophene-2,8-diyl)bis(9H-carbazole) (Compound Example 1)

Nitrogen was bubbled for 1 h into a mixture of Int. 5 (3.53 g, 5.47 mmol) and 9H-carbazole (2.01 g, 12.03 mmol) in 35 ml of xylene at 50° C. Tri-tert-butylphosphonium tetrafluoroborate (0.069 g, 0.22 mmol), palladium (II) acetate (0.024 g, 0.11 mmol) and sodium tert-butoxide (3.13 g, 32.8 mmol) were added and mixture was stirred at 160° C. overnight. Mixture was cooled down to 0° C. and quenched with 30 ml of water. Phases were separated and aqueous layer was extracted 2× with toluene. Combined organic phases were washed 2× with brine, 1× with water, dried over magnesium sulphate and concentrated under reduced pressure. The residue was purified by 2 column chromatography on silica eluting with a mixture of hexane and ethyl acetate. Fractions containing product were combined and concentrated under reduced pressure. Residue was stirred in 30 ml of hexane with 1.5 g of activated charcoal at 70° C. for 2 h and filtered. Filtrate was filtered through a silica/florisil plug (sinter funnel packed with a layer of florisil on top of a layer of silica), eluted with hexane. Filtrate was concentrated under reduced pressure. Residue was dissolved in dichloromethane, filtered and precipitated into methanol. Slurry was filtered and solid was dried in vacuum oven at 50° C. to yield the product (Compound Example 1) as a white powder (2.6 g, yield=58%). HPLC indicated a purity of 98.9%.

¹H NMR (CDCl₃) δ [ppm]−9.22 (s, 2H), 8.38 (d, 1H), 8.22 (d, 1H), 8.16 (m, 4H), 7.75 (d, 1H), 7.58 (d, 1H), 7.49 (d, 2H), 7.41 (m, 6H), 7.30 (m, 4H), 3.03 (t, 2H), 1.89 (m, 2H), 1.54 (s, 9H), 1.48 (m, 2H), 1.38 (m, 2H), 1.34-1.21 (m, 15H), 0.87 (t, 2H).

### Compound Example 2

Compound Example 2 was prepared according to the following reaction scheme:

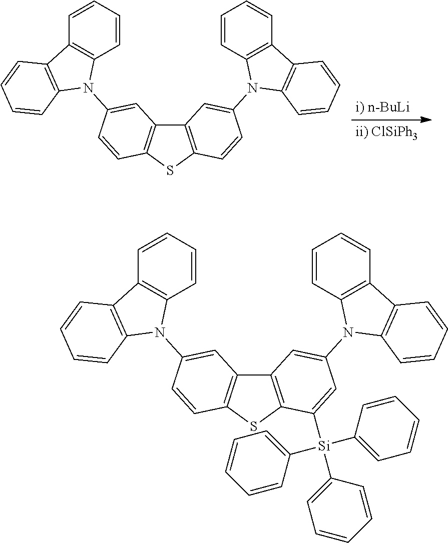 [SMILES: c1ccc2c(c1)c1ccccc1n2-c1ccc2sc3ccc(-n4c5ccccc5c5ccccc54)cc3c2c1 ; c1ccc([Si](c2ccccc2)(c2ccccc2)c2cc(-n3c4ccccc4c4ccccc43)cc3c2sc2ccc(-n4c5ccccc5c5ccccc54)cc23)cc1]

Dicarbazolyl-dibenzothiophene (36 g, 70 mmol) was dissolved in anhydrous THF (500 mL) in a dry, nitrogen-purged flask. The stirred solution was cooled to ˜−50° C. using a CO₂/MeCN cooling bath and n-butyllithium (28 mL, 70 mmol, 2.5 M) was added dropwise over ˜15 mins causing the reaction mixture to turn a deep red colour. The stirred mixture was allowed to warm to ˜−15° C. and stirred for 1.5 h at this temperature. The reaction was re-cooled to −78° C. using a CO₂/acetone cooling bath and a solution of chlorotriphenylsilane (24.75 g, 74 mmol) in anhydrous THF (50 mL) was added dropwise to give a pale red solution. The mixture was allowed to warm to room temperature overnight and quenched by addition of water. The mixture was transferred to a separating funnel and the mixtures were separated. The organics were washed with brine, dried with MgSO4, filtered and concentrated to give a white foamy solid. This crude material was purified by column chromatography on silica eluting with a heptanes/DCM mixture.

Product-containing fractions were combined and concentrated to give a white solid which was recrystallised 5 times from a toluene-acetonitrile mixture to yield the product as a white powder (25.6 g, 47%). HPLC indicated a purity of 99.86%.

¹H NMR (THF-d₈) δ [ppm]−8.75 (s, 1H), 8.64 (s, 1H), 8.14 (d, 2H), 8.08 (d, 2H), 8.05 (d, 2H), 7.83 (s, 1H), 7.77 (d, 6H), 7.67 (d, 1H), 7.46 (t, 3H), 7.39-7.46 (m, 10H), 7.34 (t, 2H), 7.30 (t, 2H), 7.22 (t, 2H), 7.18 (t, 2H).

### Compound Example 3

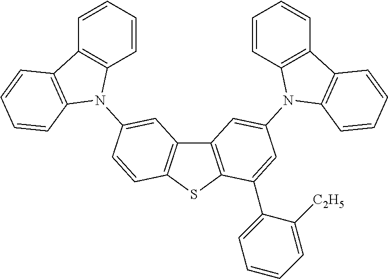 [SMILES: CCc1ccccc1-c1cc(-n2c3ccccc3c3ccccc32)cc2c1sc1ccc(-n3c4ccccc4c4ccccc43)cc12]

(2,8-di(9H-carbazol-9-yl)dibenzo[b,d]thiophen-4-yl)boronic acid (10 g, 17.9 mmol), 2-ethylbromobenzene (3.3 g, 17.9 mmol) and SPhos (130 mg, 0.32 mmol) were dissolved in a mixture of toluene (115 mL) and ethanol (15 mL). The solution was purged with nitrogen for 1 h. At the same time a solution of tetraethylammonium hydroxide (20 wt % in water, 28 mL) was also purged with nitrogen for 1 h. The base was added to the toluene/ethanol solution along with tri(dibenzylidene)dipalladium (150 mg, 0.16 mmol) and the mixture was stirred at 100° C. overnight. After cooling the reaction mixture was filtered into a separating funnel. The layers were separated and the aqueous layer was extracted with toluene. The combine organics were washed with hot water (5×50 mL), dried with MgSO₄, filtered and concentrated. The solid recrystallised from toluene/acetonitrile four times to give the product as a white solid at 99.9% purity by HPLC. The material could be purified further by sublimation.

### Compound Example 4

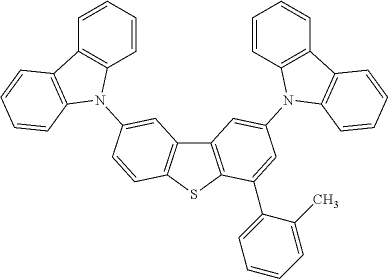 [SMILES: Cc1ccccc1-c1cc(-n2c3ccccc3c3ccccc32)cc2c1sc1ccc(-n3c4ccccc4c4ccccc43)cc12]

(2,8-di(9H-carbazol-9-yl)dibenzo[b,d]thiophen-4-yl)boronic acid pinacol ester (3.5 g, 5.5 mmol) and 2-bromotoluene (0.73 ml, 6.0 mmol) were dissolved in toluene (50 ml). The solution was purged with nitrogen for 30 minutes. At the same time a solution of tetraethylammonium hydroxide (20 wt % in water, 16 ml, 21.9 mmol) was also purged with nitrogen for 30 minutes. SPhos (49 mg, 0.11 mmol) and tri(dibenzylidene)dipalladium (50 mg, 0.06 mmol) were added to the toluene solution and the mixture was purged with nitrogen while being heated up to 105° C. The base was added to the toluene solution and the mixture was stirred at 105° C. for 20 hrs. After cooling, the layers were separated and the aqueous layer was extracted 1× with toluene. The combine organics were washed 5× with water, dried with MgSO₄, filtered and concentrated under reduced pressure. The resulting solid was dissolved in a mixture of hexane:dichloromethane (7:3) and filtered through a silica/florisil plug (sinter funnel packed with a layer of florisil on top of a layer of silica), eluted with a mixture of hexane:dichloromethane (7:3). Filtrate was concentrated under reduced pressure. The solid was recrystallised 1× from toluene/hexane and 1× from toluene/methanol to give the product as a white solid at 99.8% purity by HPLC. The material was then precipitated 4× from a dichloromethane solution into methanol, then stirred into refluxing methanol for 2 hrs, cooled down and filtered. It was dried in vacuum oven at 60° C. to give 1.56 g of Compound Example 3 at 99.8% HPLC purity, 47% yield. The material could be purified further by sublimation.

¹H NMR (CDCl₃) δ [ppm]−8.36 (d, 1H), 8.33 (d, 1H), 8.16 (m, 4H), 8.07 (d, 1H), 7.70 (dd, 1H), 7.62 (d, 1H), 7.52 (d, 1H), 7.49 (d, 2H), 7.44-7.37 (m, 9H), 7.30 (m, 4H), 2.36 (s, 3H).

HOMO levels of Compound Examples 1-3 and of Comparative Compound 1 (illustrated below) are given in Table 1.

TABLE 1

| Compound | LUMO (eV) |
|---|---|
| Comparative Compound 1 | −1.96 |
| Compound Example 1 | −2.28 |
| Compound Example 2 | −2.21 |
| Compound Example 3 | −2.08 |

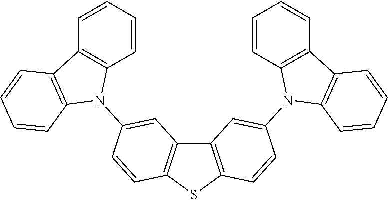 [SMILES: c1ccc2c(c1)c1ccccc1n2-c1ccc2sc3ccc(-n4c5ccccc5c5ccccc54)cc3c2c1]

### Comparative Compound 1

HOMO and LUMO levels as described herein are as measured by square wave cyclic voltammetry (CV). The working electrode potential may be ramped linearly versus time.

When cyclic voltammetry reaches a set potential the working electrode's potential ramp is inverted. This inversion can happen multiple times during a single experiment. The current at the working electrode is plotted versus the applied voltage to give the cyclic voltammogram trace.

Apparatus to measure HOMO or LUMO energy levels by CV may comprise a cell containing a tert-butyl ammonium perchlorate/or tertbutyl ammonium hexafluorophosphate solution in acetonitrile, a glassy carbon working electrode where the sample is coated as a film, a platinium counter electrode (donor or acceptor of electrons) and a reference glass electrode no leak Ag/AgCl. Ferrocene is added in the cell at the end of the experiment for calculation purposes. (Measurement of the difference of potential between Ag/AgCl/ferrocene and sample/ferrocene).

Method and settings:

3 mm diameter glassy carbon working electrode

Ag/AgCl/no leak reference electrode

Pt wire auxiliary electrode

0.1 M tetrabutylammonium hexafluorophosphate in acetonitrile

LUMO=4.8−ferrocene (peak to peak maximum average)+onset

Sample: 1 drop of 5 mg/mL in toluene spun @3000 rpm LUMO (reduction) measurement: A good reversible reduction event is typically observed for thick films measured at 200 mV/s and a switching potential of −2.5V. The reduction events should be measured and compared over 10 cycles, usually measurements are taken on the 3^{rd} cycle. The onset is taken at the intersection of lines of best fit at the steepest part of the reduction event and the baseline.

### White Device Example 1

A white organic light-emitting device having the following structure was prepared:

ITO/HIL/LEL (R)/LEL (G, B)/ETL/Cathode

wherein ITO is an indium-tin oxide anode; HIL is a hole-injecting layer comprising a hole-injecting material, LEL (R) a hole-transporting, red light-emitting layer, LEL (G, B) is a light-emitting layer containing Compound Example 1 and a blue and green phosphorescent material, and ETL is an electron-transporting layer.

A substrate carrying ITO (45 nm) was cleaned using UV/Ozone. A hole injection layer was formed to a thickness of about 65 nm by spin-coating a formulation of a hole-injection material. A red light-emitting hole transporting layer was formed to a thickness of about 17 nm by spin-coating a crosslinkable hole-transporting polymer comprising 1,4-phenylene repeat units and repeat units of formula (VII-1) endcapped with Red Phosphorescent Emitter 1 and crosslinking the polymer by heating at 180° C. The green and blue light-emitting layer was formed to a thickness of about 65 nm by spin-coating Compound Example 1 (69 wt %), Blue Phosphorescent Emitter 1 (12 wt %), Hole-transporting complex 1 (18 wt %) and a green phosphorescent emitter of tris(phenylpyridine)iridium emitter wherein each ligand is substituted with an alkylated 3,5-diphenylbenzene dendron (1 wt %). An electron-transporting layer was formed on the light-emitting layer from a polymer as described in WO 2012/133229. A cathode was formed on the electron-transporting layer of a first layer of sodium fluoride of about 3.5 nm thickness, a layer of aluminium of about 100 nm thickness and a layer of silver of about 100 nm thickness.

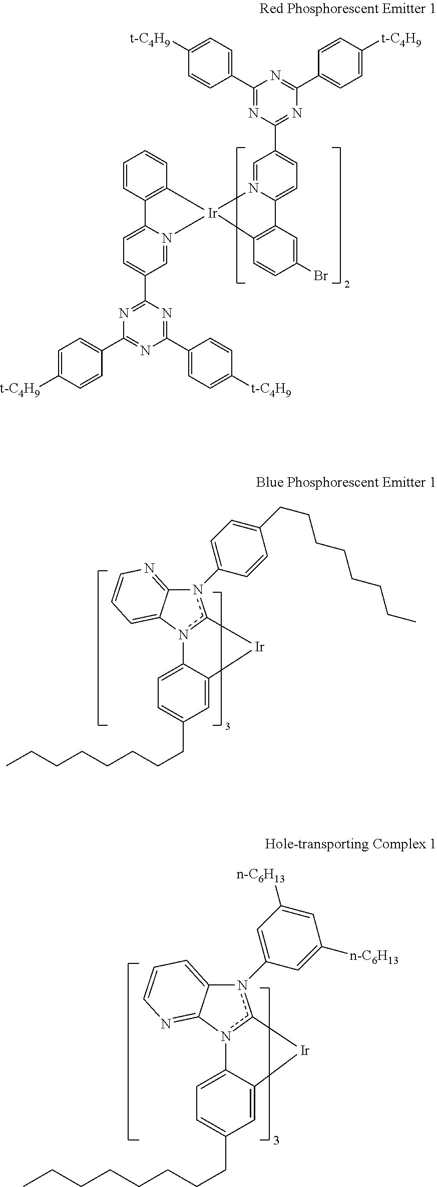

### Device Examples 2 and 3

Device Examples 2 and 3 were prepared as described for Device Example 1 except that Compound Example 1 was replaced with Compound Example 2 and 3 respectively.

### Comparative White Device 1

A device was prepared as described for White Device Example 1 except that Comparative Compound 1 was used in place of Compound Example 1.

Results are given in Table 2.

TABLE 2

| | | Voltage | | External Quantum | |
|---|---|---|---|---|---|
| | Voltage | at 1 | Efficiency | Efficiency | |
| | at 1,000 | mA/cm² | at 1,000 | at 1,000 | |
| | cd/m² | current | cd/m² | cd/m² | Max |
| | brightness | density | brightness | brightness | EQE |
| Device | (V) | (V) | (Lm/W) | (%) | (%) |
| Device | 3.9 | 3.4 | 28.9 | 17.6 | 18.3 |
| Example 1 | | | | | |
| Device | 4.9 | 4.3 | 23.6 | 15.4 | 16.2 |
| Example 2 | | | | | |
| Device | 6.0 | 5.0 | 21.1 | 16.0 | 16.6 |
| Example 3 | | | | | |
| Comparative | 6.4 | 5.6 | 18.8 | 13.6 | 14.1 |
| Device 1 | | | | | |

Although the present invention has been described in terms of specific exemplary embodiments, it will be appreciated that various modifications, alterations and/or combinations of features disclosed herein will be apparent to those skilled in the art without departing from the scope of the invention as set forth in the following claims.

1. A compound of formula (I) 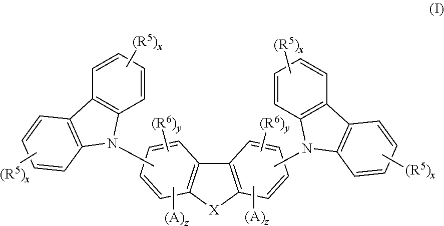
wherein:
X is O or S;
each A is a LUMO-deepening substituent;
R⁵ and R⁶ are independently in each occurrence a substituent;
x independently in each occurrence is 0, 1, 2, 3 or 4;
y independently in each occurrence is 0, 1 or 2, and
each z is independently 0 or 1 with the proviso that at least one z is 1.

2. A compound according to claim 1 wherein each A is independently selected from the group consisting of:
(Ar¹)ₚ wherein Ar¹ independently in each occurrence is a C₆₋₂₀ aryl or a 5-20 membered heteroaryl group that may be unsubstituted or substituted with one or more substituents, and p 1 or more; and
SiR¹₃ wherein each R¹ independently is a C₁₋₂₀ alkyl group or a group of formula Ar² wherein Ar² independently in each occurrence is a C₆₋₂₀ aryl or heteroaryl group that may be unsubstituted or substituted with one or more substituents.

3. A compound according to claim 2 wherein each Ar² is phenyl which may independently in each occurrence be unsubstituted or substituted with one or more substituents.

4. A compound according to claim 1 wherein each x is 0.

5. A compound according to claim 1 wherein each y is 0.

6. A compound according to claim 1 wherein one z is 1 and the other z is 0.

7. A compound according to claim 1 of formula (Ia):

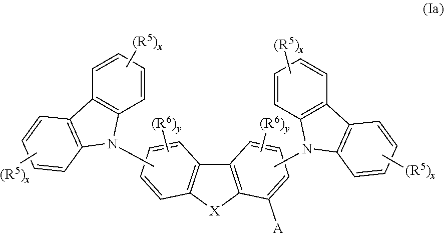

8. A compound according to claim 1 having a LUMO level of more than 2.0 eV from vacuum level.

9. A composition comprising a compound according to claim 1 and at least one light-emitting material.

10. A composition according to claim 9 wherein the light-emitting material is a phosphorescent material.

11. A composition according to claim 9 wherein the light-emitting material is a blue light-emitting material.

12. A composition according to claim 9 wherein the light-emitting material is a metal complex comprising at least one carbene group coordinated to the metal.

13. A formulation comprising a compound according to claim 1 according to at least one light emitting material and one or more solvents.

14. An organic light-emitting device comprising an anode, a cathode and a light-emitting layer between the anode and the cathode wherein the light-emitting layer comprises a compound according to claim 1.

15. An organic light-emitting device according to claim 14 wherein the organic light-emitting layer comprises a composition including at least one light emitting material.

16. An organic light-emitting device according to claim 14 wherein the device emits white light.

17. An organic light-emitting device according to claim 16 wherein the device comprises at least one further light-emitting layer.

18. A method of forming an organic light-emitting device according to claim 14 comprising the step of forming the light-emitting layer over one of the anode and the cathode and forming the other of the anode and the cathode over the light-emitting layer.

19. A method according to claim 18 wherein the light-emitting layer is formed by depositing a formulation having at least one light emitting material and one or more solvents, and evaporating the one or more solvents.

20. A polymer having a polymer backbone and comprising a group of formula (II) in the polymer backbone, as a side-chain of the polymer backbone or and end group of the polymer backbone: 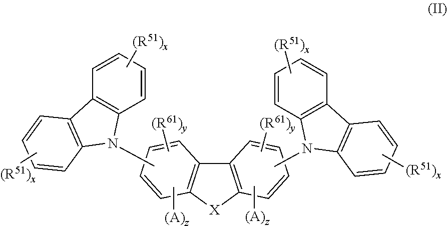
wherein X is O or S;
each A is independently a LUMO-deepening substituent;
R⁵¹ and R⁶¹ are independently in each occurrence a substituent;
x independently in each occurrence is 0, 1, 2, 3 or 4; and
y independently in each occurrence is 0, 1 or 2,
each z is independently 0 or 1 with the proviso that at least one z is 1, and
at least one of R⁵¹, R⁶¹ and A is bound to the polymer backbone.
